(19) United States
(12) Patent Application Publication
(10) Pub. No.: US 2004/0259192 A1
(43) Pub. Date: Dec. 23, 2004

(54) Method for cleaving the human growth hormone gh

(76) Inventors: Konrad Hermann, Leipzig (DE); Christoph Arkona, Leipzig (DE)

Correspondence Address: BURNS DOANE SWECKER & MATHIS L L P, POST OFFICE BOX 1404, ALEXANDRIA, VA, 22313-1404, US

(21) Appl. No.: 10/472,472

(22) PCT Filed: Mar. 9, 2002
(86) PCT No.: PCT/EP02/02606

(30) Foreign Application Priority Data
Mar. 20, 2001 (DE) 101 13 604.8

(51) Int. Cl. A61K 38/48; C12P 21/06

(52) U.S. Cl. 435/68.1; 424/94.63

(57) ABSTRACT

The invention relates to a method for cleaving human growth hormone GH by means of matrix metalloproteinase MMP. It has been found that MMP-3 cleaves the growth hormone into two fragments, the 16 kDa fragment of which is stable. The invention solves the problem of providing means which positively influence both tumor growth, proliferative diabetic retinopathy and others as well as angiogenesis, in particular on treatment the cardiac infarction, retarded wound healing, menstrual cycle disturbances and others, by means of regulation of the human growth hormone.

This invention relates to the detection of a novel substrate not previously described in the literature for the human matrix metalloproteinase. The knowledge of the cleavage of this substrate by MMP shall be employed in methods for the production of pharmaceutical preparations and the use of MMP inhibitors and MMP inductors as a medicament for treating diseases in humans and animals.

The Human Growth Hormone (GH)

Human GH is a 22 kDa peptide hormone composed of a single polypeptide chain. There are two disulfide bridges between the amino acids cysteine 53 and cysteine 165 or cysteine 182 and cysteine 189. GH is a member of the human somatotropin family which shows a number of growth-inducing, differentiating and metabolically regulating effects in the most different tissues (1). The somatomedines and the insulin-like growth factors I and II are formed in the liver under the direct influence of GH. In addition to the direct effect of GH, these factors are co-responsible for the growth hormone effect, in particular in cartilage, osteoepiphyses, fatty tissue and muscles.

In addition, GH is the main regulator for postnatal somatic growth (2). It is required for sex differentiation, puberty, gametogenesis and lactation (8).

The precise GH response depends on the respective cell type and the regulative overall situation in which the hormone acts. The hormone effect is initiated by its interaction with the GH receptor or with a receptor from the GH receptor family. In the further course, GH modulates the gene expression via the concentration or activity of the most different transcription factors (18).

GH stimulates the longitudinal growth of bones by directly influencing the chondrocytes of the epiphyses (3). It was also possible to show that in osteoblasts GH stimulates the synthesis of the MMP-9 and MMP-2 collagenases (15). GH also plays a role both in vitro and in vivo via an induction of the proliferation of preadipocytes and their differentiation with respect to adipocytes (4). It supports the lipolysis by directly inhibiting the lipoprotein lipase (10).

GH also stimulates the chemotaxis of monocytes (5). In this connection, the stimulation of the actin reorganization in the cytoskeleton and the polymerization of the microtubuili seem to be of special significance (6,7).

As compared to a healthy control group GH-deficient rats showed a significantly reduced mRNA expression for type I collagen, type III collagen and insulin-like growth factor-I (IGF-I) in fibroblasts, which returned to normal after an extreme GH administration (11). These data prove that fibroblasts are also important target cells for the GH action. The findings by Lal et al. (12) seem to be correlated therewith. They were able to prove that in burn patients the wound healing period is significantly shortened after a GH administration.

In the past few years, numerous experimental publications also proved the significance of GH within the regulative network of the central nervous system. For example, it was found that mental capacity, psychological profile, memory, motivation and working capacity improved substantially after the administration of GH in patients having a reduced GH level.

The regulative role of GH is reduced during the “acute phase response” in inflammations. In this connection, it was also possible to show that GH modulates the adhesion of neutrophil granulocytes during the inflammatory response via a tyrosine phosphorylation of intracellular targets and is thus relevantly involved in the adhesion and extravasion of neutrophils (13). As compared to the healthy control group patients with GH deficiency show a significantly increased plasma level of fibrogen, plasminogen activator inhibitor type 1, acute phase protein and the soluble adhesion molecules sE-selectin, sP-selectin and sICAM-1 which returned to normal after a GH substitution therapy (19).

GH has a proliferation-stimulating effect on many cell types of the human body. Along with wound healing this effect seems to be of decisive significance in the permanent regeneration of the intestinal epithelium. This proliferative effect might also cause the increased incidence of neoplastic polyps in patients undergoing a GH therapy (14).

Recently, the Garcia-Ruiz' study group was able to prove with transgenic mice expressing the fusion gene of bovine GH that similar histologic changes as typical of human arthritis, were detectable in the joints of the animals. Interestingly enough, these animals also had autoantibodies against single-stranded and double-stranded DNA and antinuclear antibodies (ANA) which are typical of human rheumatoid arthritis (16). Denko et al. (20) discovered that the GH level is significantly raised in the synovial fluid and in the serum of rheumatics. This is a finding which the authors consider to be significant in the pathophysiology of arthritic diseases. The findings of Yamashita et al. (17) who were able to show that GH proliferatively stimulates the human Th cell clones, Th0 and Th2, while Th1 is not influenced, show the same trend of immunomodulation by GH.

At present, the effect of GH as an angiogenesis-stimulating signal is well proved (33, 34, 35, 36). From a physiological point of view, this vessel-stimulating effect plays an extremely important role during organogenesis and fetal and infantile growth. In wound healing this angiogenetic effect of GH is an essential precondition for rapid wound closure (12). One of the most dreaded side-effects of the poorly stabilized diabetes mellitus type I is the proliferative diabetic retinopathy which is characterized by a hyperproliferation of the microvascular endothelial cells of the retina. As a result of a reduced function of the hypophysis or an operative removal thereof, the proliferative retinopathy was reduced or eliminated in these patients. At the end of the 60ies, the hypophyses of over 900 patients suffering from a proliferative diabetic retinopathy were removed for this reason. Rymaszewski et al. (33) proved by way of experiment that this observed therapeutic effect was due to a reduced GH level in the organism. They were able to prove the stimulating influence of GH on the proliferation of retinal endothelial cells.

Struman et al. (37) recently published data which confirmed this stimulating effect of GH on endothelial cells and also proved that an about 16 kDa N-terminal cleavage product of human GH significantly inhibited the proliferation of microvascular endothelial cells depending on the dosage. The authors obtained this cleavage product by proteolytic cleavage of native GH by means of plasmin, thrombin or subtilisin or recombinantly by means of expression in E. coli. The angiogenesis assays conducted in vitro with GH and the 16 kDa cleavage product greatly confirmed the proliferation experiments. GH markedly induced the blood vessel formation and the 16 kDa cleavage product of GH inhibited significantly angiogenesis. As early as in 1993 Warner et al. (38) described by means of the Western blot technique the occurrence of a 17 kDa variant of GH in the serum which in the morning and evening was at a concentration ratio to normal 22 kDa GH of 80:20.

Angiogenesis is essential for many physiological and pathological processes. Here, the formation and reorganization of the embryonic tissue, the development of the placenta, the wound healing and the tumor growth are to the fore. Angiogenesis is an upregulated controlled and balanced process from proangiogenic and antiangiogenic regulatory signals and is always connected with the enzymatic action of proteases and MMPs.

The Human Matrix Metalloproteinases (MMPs)

The human MMPs belong to a constantly growing enzyme family having over 20 members to date. Regarding the structure, the individual MMP types are closely related to Zn²⁺- and Ca²⁺-containing neutral endopeptidases whose main substrates were initially collagen and related proteins of the extracellular matrix. This enzyme group plays an important role in the metabolism of the connective tissue, such as morphogenesis, tissue resorption and tissue remodeling, nerve growth, reproduction, hair follicle development, platelet aggregation, macrophage and neutrophil functions, inflammation, cell migration and angiogenesis. The involvement of MMPs in pathological processes, such as rheumatoid arthritis, osteoarthritis, tumor invasion and tumor metastases, ulcerations, peridontal diseases, fibroses, atherosclerosis and aortic aneurysm, are well documented (21).

The MMPs are expressed as inactive preproenzymes and secreted as soluble inactive proenzymes in the extracellular compartment or incorporated into the plasma membrane of the cell surface as membrane-bound MMPs. A common characteristic of MMPs is their modular structure from homologous domains. The modular structure comprises the signal peptide (about 20 amino acids), a propeptide (about 80 amino acids), a conserved zink-binding catalytic domain (about 170 amino acids), a linker domain, a hemopexin/vitronectin-like domain (about 210 amino acids), fibronectin type II repeats (about 75 amino acids) and in the case of the membrane-bound MMPs either a transmembrane domain or a cytoplasmic domain (about 80-100 amino acids) or a glycophosphatidyl inositol anchor in the case of MMP-17 (22, 23).

The activity of MMPs is finely balanced in vivo by different mechanisms of activation and inhibition. The expression of MMPs is induced by growth factors, cytokines, hormones and by the interaction of the cells with components of the extracellular matrix. The proenzymes are activated in vivo via the proteolytic cleavage of the propeptide by numerous proteases (inter alia trypsin, α-chymotrypsin, cathepsin G, plasmin, thrombin, leukocyte elastase, kalikrein) by autoactivation or via an MMP activation cascade, the focus of which is MMP-14. There exist in vivo four natural proteinogenous inhibitors, tissue inhibitor of matrix metalloproteinases (TIMPS) which can efficiently block the proteolytic activity of MMPs. In normal tissue, the activity of MMPs is very low because the major part thereof are available as inactive proenzymes and are only activated when required and in accordance with the physiological conditions are very rapidly inactivated again. A disturbance of this balance results in a plurality of diseases and pathological symptoms according to today's knowledge.

Matrix metalloproteinase-3 (MMP-3, stromelysin-1, EC 3.4.24.17)

The first report on the enzymatic activity of this enzyme was given as a proteoglycan-degrading metalloproteinase from cartilage (27) or as a neutral proteinase from rabbit fibroblasts (28) in 1974. The enzyme was isolated from the cell culture supernatants of rabbit bone cells and prepared in a biochemically pure way and referred to as proteoglycanase (29). In 1985, Chin et al. (30) changed the name of this enzyme to stromelysin. In 1986, Okada et al. (31) purified two isoforms of the enzyme having a molecular weight of 45 kDa and 28 kDa from the culture supernatant of human synovial cells from patients suffering from rheumatoid arthritis and described these enzymes as MMP-3.

The Human MMP-3 Gene is Localized on Chromosome 11q22-q23 (32).

Human pro-MMP-3 consists of a propeptide (82 amino acids), a catalytic domain (165 amino acids), a linker domain rich in proline (25 amino acids) and a C-terminal hemopexin/vitronectin-like domain (188 amino acids) (24). The zinc-binding motif (HEXXHXXGXXH) (25) is found within the catalytic domain. The enzyme has two zinc and two calcium ions. The pH optimum for this MMP is at pH 5.5-6.0 with a marked activity shoulder at pH 7.5-8.0. The presence of calcium is necessary to obtain the active conformation of the enzyme.

In normal tissue, MMP-3 is secreted by the following cell types:

Fibroblasts, chondrocytes, osteoblasts, macrophages, proliferating basal keratinocytes, lipocytes, microvascular endothelial cells, smooth muscle cells, mamma cells, endometrium cells in the menstrual phase and placental mesenchymal cells, retinal pigment cells.

MMP-3 can be identified in the below pathological tissues in a highly increased activity: osteoarthritic cartilage, synovial membrane and serum from RA patients, activated E-lymphocytes in the synovial membrane in the case of RA, connective tissue in the case of wound healing, inflammatory intervertebral disks, cholesteatoma epithelium, atherosclerotic plaques, epithelial cells of the respiratory tract following wounding, aortic aneurysm, gastrointestinal ulcerations, Crohn's disease, colorectal carcinoma, squamous cell carcinoma, bronchial carcinomas and pulmonary and esophageal carcinomas.

MMP-3 cannot convert its own inactive proforms into the active enzyme but is capable of activating pro-MMP-8, pro-MMP-9 and pro-MMP-13.

MMP-3 cleaves different components of the extracellular matrix but not the triple helical regions of the native interstitial collagen. In contrast to MMP-1, MMP-3 binds to the interstitial type I collagen without cleaving it (26). Table 1 lists summarily all of today's known natural human substrates for MMP-3.

TABLE 1
Natural human substrates of human MMP-3

| Substrate | Origin |
|---|---|
| Aggrecan core protein | human |
| Cartilage link protein | human |
| Fibronectin | human |
| α₂-macroglobulin | human |
| α₁-proteinase inhibitor | human |
| α₁-antichymotrypsin | human |
| Pro-MMP-1 | human |
| Pro-MMP-7 | human |
| Pro-MMP-8 | human |
| Pro-MMP-9 | human |
| Pro-MMP-13 | human |
| Substance P | human |
| Insulin β-chain | human |
| IGF-BP-3 | human |
| SPARC (BM-40, osteonectin) | human |
| Fibrin γ-chain | human |
| Vitronectin | human |
| Tenascin | human |
| Perlecan | human |
| Laminin | human |
| Elastin | human |
| Interleukin 1β | recombinant, human |

The natural substrates known to date have been found and characterized in vitro without exception. However, little is known about the actual function/situation in vivo. All of the substrates listed herein are not only detected and processed by MMP-3 but also represent substrates for other MMPs. The only reason for an assumed substrate specificity is often only the different affinities for the substrate and in the respective cleavage site.

Nowadays it is assumed concurrently that the in vivo relevant substrates are only known to some extent for the entire MMP group. The characterization of specific substrates, i.e. of proteins, which are only cleaved by one MMP will induce a considerable insight gain in this field. The detection of new substrates for the enzymatic action of MMPs will not only create a more thorough insight into the actual function of MMPs in vivo but will also yield new targets for innovative therapeutic strategies.

Biological Significance of the Specific Cleavage of Human GH by MMP-3

According to today's knowledge, the specific cleavage of GH by MMP-3 is physiologically significant mainly because of the opposite regulation of angiogenesis by the native hormone (proangiogenic) or by its 16 kDa fragment (antiangiogenic).

This regulation mechanism is most obvious in wound healing, since in this case the vessel growth in the granulation tissue has to be stimulated in a close connection in terms of time, followed by the inhibition thereof.

The studies conducted by Young and Grinnell (39) who determined the concentration of MMPs in the wound fluid of burn patients in a longitudinal section in terms of time prove that it is not all of the MMPs that have their main function in the degradation of matrix proteins. For example, it was possible to prove that MMP-9 was detectable in the wound fluid only 4-8 hours after the burn and the peak of the activated enzyme occurred between day 0 and day 2. In contrast thereto, MMP-3 was first observed on day 4 after the injury, a finding proving that MMP-9 has mainly degenerative tasks (degradation of the destroyed connective tissue) and MMP-3 is inter alia responsible for functions not detected to date. One of these functions might be e.g. the specific cleavage of GH in the wounded area and the associated reduction of angiogenesis. By means of the well-calculated activation or inhibition of MMP-3 it might thus be possible to carry out a treatment strategy adapted to the individual patient for non-healing wounds.

The activating influence of GH on the cardiac muscle growth and the cardiac function, in particular on the contractile force of the myocardium is well proved and was used clinically with success in patients suffering from GH deficiency (40, 41, 42). On the other hand, patients suffering from a GH overproduction have massive cardiac problems such as myocardial hypertrophy and in particular the hyperkinetic syndrome (40). Experimental animals with experimental cardiac infarction showed an improved action of the heart after the administration of GH. In the first 6 hours after an infarction, patients suffering from an infarct had an about 3 times higher GH concentration than 12 weeks after the infarction (42). The repair process of the myocardium after an infarction is a highly complex process of manifold signals of the inflammation cascade, the remodeling of the extracellular matrix, the release of multiple neuro-humoral stimuli and the adaptive response of the myocardium cells on these signals. Directly after the infarction, non-specific inflammatory processes and the proteolytic degradation of the infarcted muscle initially play the main role. According to Heyman et al. (43) the proteases urokinase-type plasminogen activator (u-PA), MMP-9 and MMP-3 are of decisive significance in this process which often results in a rupture of the myocardium. When the gene was eliminated for u-PA and the MMPs were inhibited by TIMP-1, it was possible to fully prevent a myocardium rupture in animal experiments as a delayed reaction of the experimental cardiac infarction. In addition, the authors observed in the u-PA-deficient and MMP-9-deficient animals a significant reduction in angiogenesis. Similar to the wound healing example it was recently possible to show that directly after the infarction MMP-9 is induced and expressed while MMP-3 was not detected until two days after the infarction (10). In this connection, it can be derived that anabolic GH and its 16 kDa fragment also belong to the cycle of neoangiogenesis and that an inhibition or activation of MMP-3 has useful therapeutic effects in this important group of diseases. The specific inhibition of MMP-3 by suitable inhibitors would inter alia support the stimulating influence of GH on the healing of the area of infarction, in particular on the stimulation of the myocardium cell activity and an enhanced angiogenesis and, on the other hand, have no decisive influence on the degradative processes (tissue degradation, tissue remodeling).

The female menstrual cycle is closely related to angiogenic and antiangiogenic regulation mechanisms. As early as in 1988 Kennedy and Doktorcik (45) described that GH stimulates the proliferation of the uterus and its cellular growth. Increased GH concentrations also seem to be causally related to the occurrence of uterine carcinomas (46, 47).

MMPs are also important for the menstrual cycle. For example, Rodgers et al. (48) were able to prove by means of in situ hybridization that numerous MMPs are regulated differently during the menstrual cycle. MMP-3 cannot be identified in the proliferative phase in the epithelium and can only be identified to a very minor extent in the stroma. The enzyme cannot be detected in the secretory and in the late secretory phases. On the contrary, the stroma in the menstrual phase shows strong mRNA expression for MMP-3. This discovered expression pattern for MMP-3 is also useful under physiological aspects. The lacking or only very weak MMP-3 expression in the proliferative and secretory phases effects that GH is not cleaved and can thus exert its anabolic regulatory effect, in particular the activation of angiogenesis. In the menstrual phase which does not require any angiogenesis-supporting signals, MMP-3 is expressed and cleaves GH into its 16 kDa fragment which exerts an antiangiogenic effect. Therefore, it can be assumed that in the case of menstrual problems which are not associated with hormones, the expression of MMP-3 is dysregulated and, as a result, GH is also fragmented by MMP-3 in phases other than the menstrual one. This would lead to a disturbance of the physiological regulation of angiogenesis. A specific inhibition of MMP-3 in these phases might thus normalize the physiological balance of GH and MMP-3 and be considered a new therapeutic principle for this group of diseases.

It is thus the object of this invention to correlate the degradative action of MMPs with GH important for angiogenesis. A further object of the present invention is the detection of a completely new natural substrate for MMP-3 and the presentation of its regulative effect on the proliferation of microvascular endothelial cells and angiogenesis. It is another object of this invention to present the physiological and pathophysiological significance of the specific cleavage of human GH by human MMP-3 and to formulate new therapeutic approaches for the treatment of diseases on this basis.

The invention is based on the scientific knowledge of identifying a novel substrate not described in the literature to date for the human matrix metalloproteinase-3 (MMP-3, stromelysin-1). The now available knowledge of the cleavage of this substrate by MMP-3 shall be employed in processes for pharmaceutical preparations and the use of MMP inhibitors and MMP inductors as medicaments for treating diseases in man and animals in which the inhibition or activation of human MMP-3 supports neovascularization or prevents it by the newly discovered cleavage mechanism.

By means of two independent methods it was possible to show that both the recombinant catalytic domain of human MMP-3 and the full-length enzyme bind to the human growth hormone (GH) and cleave it into a stable 16 kDa fragment and into an instable 6 kDa fragment. Using automatic protein sequencing and MALDI-TOF MS it was possible to identify the cleave site in GH and to prove the amino acid sequence of the 16 kDa cleavage product. The identified cleavage of GH is selective for MMP-3 since other MMPs (MMP-2, MMP-8, MMP-9, MMP-14) had no, or only minor, degradative effects on human GH.

The above object is achieved according to the claims. The dependent claims relate to advantageous embodiments and uses of the invention.

The invention is explained in more detail by the below examples:

### EXAMPLE 1

Identifying the assumed interaction between human MMP and the human growth hormone (GH) by means of the yeast two-hybrid systems.

Components of the commercial LexA-Two hybrid system from Clontech company were used to achieve this object.

The cDNA of human pro-MMP-9 (gelatinase B; EC 3.4.24.35) was cloned into the yeast expression vector pEG 202. This construct was then transformed into the yeast strain EGY 48/pSH 18-34. This thus transfected yeast then expresses on suitable media the human MMP-9 with N-terminal fusion of the LexA protein which represents a transcription factor derived from bacteria.

Having checked the correct expression of the LexA-MMP-9 fusion protein by means of known Western blot technique, a purchasable cDNA library from human placenta (Clontech HL4506AK) was transformed into this yeast clone. This cDNA library expresses the proteins as N-terminal fusion with what is called the B 42 activation domain.

By selection for amino acid-deficient medium, yeast clones were then selected which

1. are capable of autonomous synthesis of leucine and

2. simultaneously express β-galactosidase.

Both criteria together indicate that a protein derived from the cDNA library and interacting with MMP-9 is expressed in the yeast.

The clones thus obtained were subjected to further tests as regards the reproducibility and specificity of the identified interaction.

In this way it was possible to identify by DNA sequencing a clone which had the following amino acid sequence:

R-K-D-M-D-K-V-E-T-F-L-R-I-V-Q-C-R-S-V-E-G-S-C-G-F

A computer analysis of the amino acid sequence resulted in 100% conformity with the C-terminus of the human hypophysis growth hormone.

It was tested in an informing preliminary test whether MMP-9 can cleave human GH in vitro. As shown in Example 2, GH was in this case incubated with activated MMP-9 and subjected to PAA gel electrophoresis. In spite of long incubation periods and different enzyme concentrations it was not possible to prove cleavage of GH by MMP-9. Since MMP-9 resembles MMP-3 as regards its domain structure, it should then be checked whether human MMP-3 can cleave human GH.

### EXAMPLE 2

Cleavage of Human Hypophysis Growth Hormone (GH) by the Recombinant Catalytic Domain of MMP-3 or the Full-Length Form of MMP-3

Human GH (SIGMA S-4776) was incubated with the recombinant catalytic domain of MMP-3 or with the activated full-length enzyme MMP-3 (EC 3.4.24.17) at a molar ratio of 50:1 at 37° C. overnight. 100 mM Tris-HCl, pH 7.5/100 mM NaCl/10 mM CaCl₂/0.05% (w/v) Brij 35 served as incubation buffer. The following samples served as a control:

1. GH incubated with the same volume of buffer instead of MMP-3,

2. GH incubated with MMP-3 in the presence of 1 mM 1,10-phenanthroline (SIGMA P-9375) as MMP inhibitor.

The reaction is stopped by the addition of ¼ volume of 5 times reducing Laemmli buffer and incubation at 90° C. for 4 minutes. The samples were subsequently separated on a 15% SDS polyacrylamide gel according to a generally known method. The proteins were prepared by means of coomassie or silver staining.

As follows from FIG. 1, the 22 kDa GH is cleaved by MMP-3 into two fragments of 16 kDa and 6 kDa. GH incubated with buffer instead of MMP-3 or in the presence of the MMP inhibitor phenanthroline was not cleaved. Whenever in place of the specific MMP inhibitor, phenanthroline, the general cysteine protease inhibitor E-64 (trans-epoxysuccinyl-L-leucylamido-(4-guanidino)butane, SIGMA E-3132), the asparatate protease inhibitor pepstatin A (isovaleryl-Val-Val-Sta-Ala-Sta [Sta=statine=(3S,4S)-4-amino-3-hydroxy-6-methylheptanoic acid], SIGMA P-4265) or a cocktail of serine and cysteine protease inhibitors (complete™, EDTA-free Boehringer Mannheim) is added to the test batch, no influence of the GH cleavage by MMP-3 was observed.

### EXAMPLE 3

Immunological Detection that the 16 kDa Fragment Belongs to Human GH

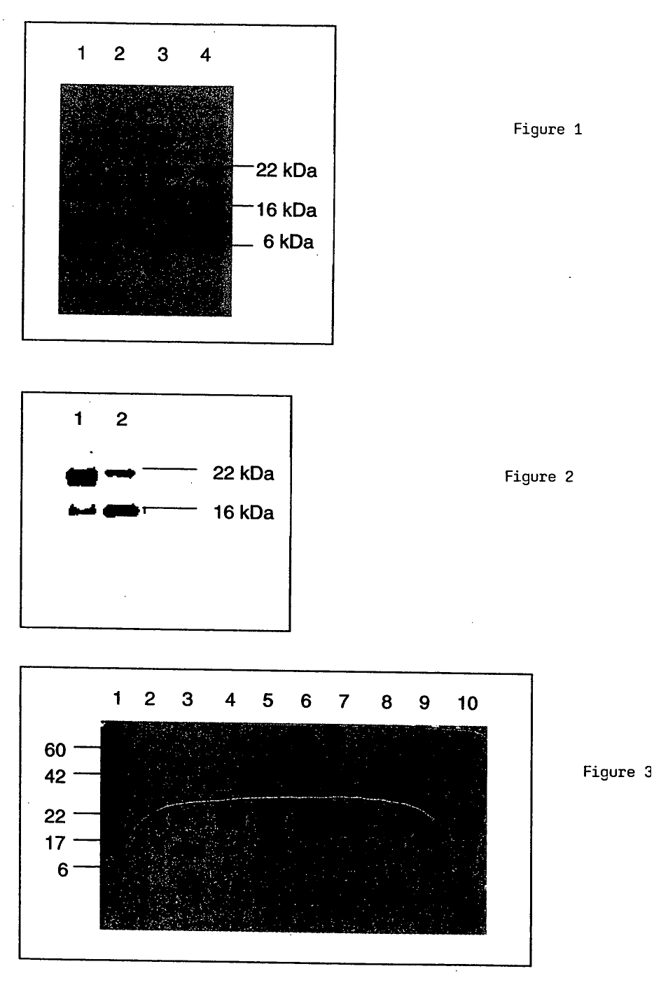

According to the generally known method of immunoblot (Western blot) with the aid of a polyclonal antibody (Dr. J. Kratzsch, University Leipzig) against human GH it was possible to show beyond any doubt that the two GH fragments from the hypophysis having molecular weights of 16 kDa and 6 kDa were recognized by the antibody and stained specifically (FIG. 2). These studies served for verifying that the fragments shown in SDS-PAGE of 16 kDa and 6 kDa are immunologically identical and thus derived from human GH or represent the specific cleavage products thereof.

### EXAMPLE 4

Detection of the Specificity of Human GH Cleavage by MMP-3

In accordance with the method shown in Example 2, other human MMPs were also tested for checking the specificity of the GH cleavage by MMP-3. As shown in FIG. 3, only MMP-3 cleaves GH into the typical 16 kDa fragment while MMP-8, MMP-9 and MMP-14 showed no degrading effect. The findings as shown prove experimentally that of the tested human MMPs only MMP-3 can cleave effectively into its fragments of 16 kDa and 6 kDa and thus has to be regarded as specific.

### EXAMPLE 5

Determination of the Cleavage Site of Human GH by MMP-3

In order to determine the cleavage site within GH following MMP-3 cleavage, the 16 kDa fragment was analyzed by means of Edman degradation and MALDI-TOF MS.

The N-terminal sequence of the 16 kDa fragment was carried out with a protein sequencer 473A from Applied Biosystems according to the known protocol. Based on the N-terminal end of the peptide chain, in this case one amino acid after the other is cleaved by phenylisothiocyanate. The individual amino acids are determined sequentially by means of HPLC.

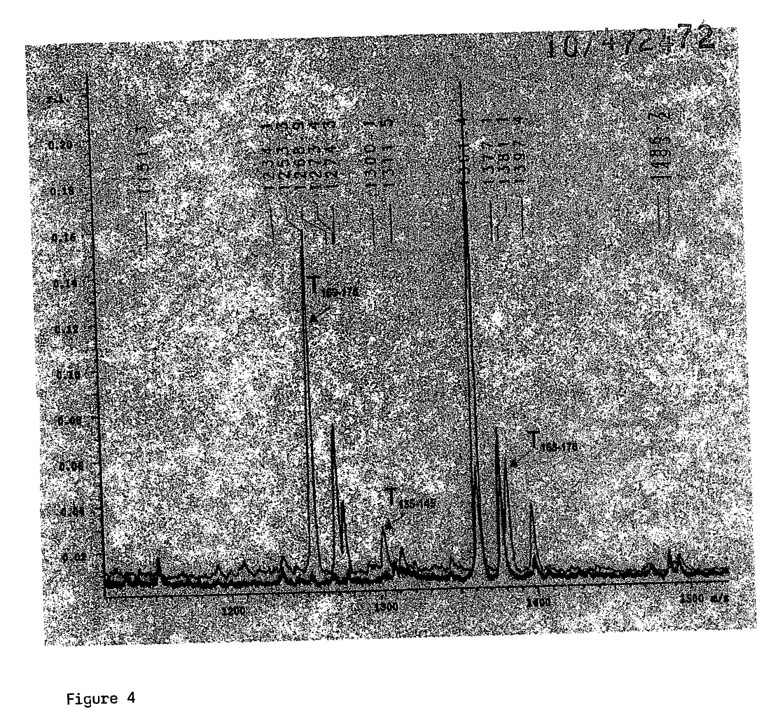

MALDI-TOF MS (matrix-assisted laser desorption ionisation-“time of flight”-mass spectrometry) can determine the molecular weight of peptides with high accuracy (at least 0.1°). Both the intact 22 kDa GH and the 16 kDa fragment derived therefrom were subjected separately to tryptic degradation. The accurate molecular masses of all tryptic peptides were analyzed by means of MALDI-TOF MS. Based on the known primary sequence of GH, it was then possible to attribute each mass peak obtained in MALDI-TOF MS to a tryptic fragment. A comparison of the results of the intact 22 kDa GH with the 16 kDa fragment shows a peptide (FIG. 4, T₁₃₅₋₁₄₅) which occurs in the 22 kDa GH but lacks from the 16 kDa fragment. Thus, amino acid 135 is the amino acid in front of which the 16 kDa fragment breaks, i.e. the amino acid R 134 is the C-terminal amino acid of the 16 kDa fragment. These analyses were carried out using the Biflex™ III device of Bruker Saxonia company.

Together with the results of the Edman degradation it turned out that the 16 kDa fragment comprises the amino acids F1-R134 of human GH.

### EXAMPLE 6

Influence of 22 kDa GH and the 16 kDa Fragment on the Proliferation of Human Microvascular endothelial cells

Human microvascular endothelial cells (HUVEC) of the 3rd passage were kindly provided by Dr. Jurgen Salvetter (Leipzig University). These cells were prepared according to the information furnished by Norman and Karasek (49). HUVEC were used up to the 9^{th} passage for the proliferation experiments. HUVEC were tested up to confluence in RPMI 1640 medium with additions of 10% (v/v) tested endotoxin-deficient fetal calf serum (Biochrom), 2 mM L-glutamine (GIBCO), 25 μg/ml endothelial cell growth supplement (SIGMA), 50 μg/ml streptomycin plus 50 units/ml penicillin (Life Technologies). The confluent cells were harvested by means of 5% trypsin/0.2% EDTA (BIOCHROM) at 37° C. for 5 minutes. A suspension of 10,000 cells in RPMI 1640 medium plus 2% (v/v) tested endotoxin-deficient fetal calf serum (Biochrom), 2 mM L-glutamine (GIBCO), 50 μg/ml streptomycin plus 50 units/ml penicillin (Life Technologies) were transferred to 24-well plates coated with 30 μg/ml type I collagen from calf skin (IBFB). The cells were cultured with 5% CO₂ at 37° C. and 95% humidity for 12 hours, the medium was changed and 20 ng/ml human recombinant bFGF (BIOCHROM) as positive control, 40 ng/ml human GH (SIGMA) and 40 ng/ml 16 kDa fragment of human GH (purified after cleavage of human GH by MMP-3) were added to 4 wells each. The cells were washed with the same medium after 24 hours of incubation and removed using trypsin/EDTA and the cell number was determined in a Coulter Counter. All the data are average values±standard deviation and represent quadruple analyses of a test batch which is however representative of the other 5 independent analyses.

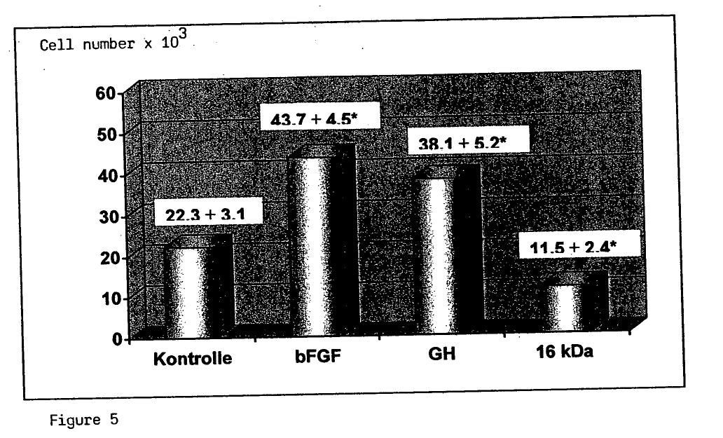

It is evident from FIG. 5 that as expected bFGF stimulates the proliferation of HUVEC. GH also shows a marked proliferative effect on these cells which, however, does not fully achieve the level of bFGF. In contrast thereto, the 16 kDa fragment of human GH inhibits significantly the proliferation of HUVEC.

By means of these data it was possible to prove that the 16 kDa fragment has an effect on HUVEC contrary as compared to native 22 kDA GH.

Substances Having an MMP-3-Activating Effect

3-(3-mercaptopropyl)-1-methyl-(1H,3H)quinazoline-2,4-dione

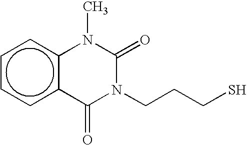

activation of MMP-3 by about 20%

3-(2′-mercaptoethylamino)-2H-1,2,4-benzothiadiazine-1,1-dioxide

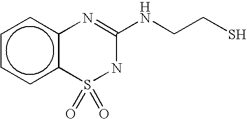

activation of MMP-3 by about 20%

substances having an MMP-3-inhibitory effect

(R,S)-8-chloro-2-mercaptomethyl-2,3-dihydro-thiazolo[2,3-b]quinazoline-5-one

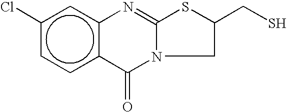

80% inhibition with 10 μM

(R,S)-1-(2′,3′-dimercapto-prop-1′-yl)quinazoline-2,4(1H,3H)-dione

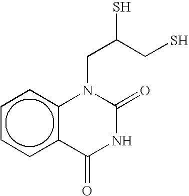

75% inhibition with 4.0 μM

3-(2-mercaptopropyl)quinazoline-2,4(1H,3H)-dione

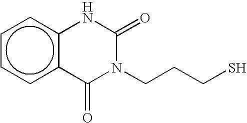

60% inhibition with 10 μM

(R,S)-2-mercaptomethyl-2,3-dihydro-thiazolo[2,3-b]quinazoline-5-one

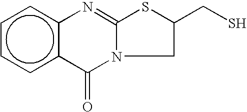

50% inhibition with 10 μM

(R,S)-2-mercaptomethyl-2-methyl-2,3-dihydro-thiazolo[2,3-b]quinazoline-5-one

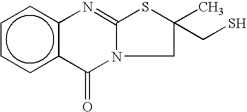

60% inhibition with 10 M

### Literature

1. Isaksson OGP, Eden S, Jansson JO. (1985) Annu Rev Physiol 47, 483-499

2. Butler A A, Ambler G R, Breier B H, LeRoith D, Roberts C T Jr., Gluckman P D. (1994) Mol Cell Endocrinol 101, 321-330

3. Ohisson C, Nilsson A, Isaksson O, Lindahl A. (1992) Proc Natl Acad Sci USA 89, 9826-9830

4. Wabitsch M, Heinze E. (1993) 40 5-9

5. Wiedermann C J, Reinisch N, Braunsteiner H. (1993) Blood 82, 954-960

6. Goh E L, Pircher T J, Wood T J, Norstedt G, Graichen R, Lobie PE. (1997) Endocrinology 138, 3207-3215

7. Goh E L, Pircher T J, Lobie PE. (1998) Endocrinology 139, 4364-4372

8. Hull K L, Harvey S. (2000) Rev Reprod 5, 175-182

9. Nyberg F (2000) Front Neuroendocrinol 21, 330-348

10. Carrel A L, Allen D B. (2000) J Pediatr Endocrinol Metab 13, Suppl 2, 1003-1009

11. Wilson V J, Rattray M, Thomas C R, Moreland B H, Schulster D. (1998) Growth Horm IGF Res 8, 431-438

12. Lal S O, Wolf S E, Herndon D N. (2000) Growth Horm IGF Res 10, Suppl B, S39-43

13. Ruy H, Lee J H, Kim K S, Jeong S M, Kim P H, Chung H T. (2000) J Immunol 165, 2116-2123

14. Shulman D I. (2000) Endocrine 12, 147-152

15. Rousselle A V, Damiens C, Guicheux J, Pilet P, Padrines M, Heymann D. (2000) Rev Chir Orthop Reparatrice Appar Mot 86, 256-264

16. Ogueta S, Olazabal I, Santos I, Delgado-Baeza E, Garcia-Ruiz JP. (2000) J Endocrinol 165, 321-328

17. Yamashita N, Hashimoto Y, Honjo M, Yamashita N. (2000) Life Sci 66, 1929-1935

18. Rastegar M, Rousseau G G, Lemaigre F P. (2000) Endocrinol 141, 1686-1692

19. Kvasnicka J, Marek J, Kvasnicka T, Weiss V, Markova M, Stepan J, Umlaufova A. (2000) Clin Endocrinol (Oxf) 52, 543-548

20. Denko C W, Boja B, Moskowitz R W. (1996) Osteoarthritis Cartilage 4, 245-249

21. Matrix Metalloproteinases. Eds. Parks W C, Mecham RP (1998) Academic Press; San Diego, London, Boston, New York, Sydney, Tokyo, Toronto

22. Nagase H, Woessner J F. J Biol Chem 274, 21491-21494

23. Itho Y, Kajita M, Kinoh H, Mori H, Okada A, Seiki M. (1999) 274, 34260-34266

24. Nagase H. pp. 43-84 in Matrix Metalloproteinases Eds. Parks W C, Mecham RP (1998) Academic Press; San Diego, London, Boston, New York, Sydney, Tokyo, Toronto

25. Bode W, Gomis-Ruth F X, Stocker W. (1993) FEBS Lett 331, 134-140

26. Murphy G, Allan J A, Willenbrock F, Cockett M I, O'Shea M, Docherty A J P. (1992) J Biol Chem 267, 9612-9618

27. Sapolsky A I, Howell D S, Woessner J F Jr. (1974) J Clin Invest 53, 1044-1053

28. Werb Z, Reynolds J J. (1974) Biochemistry 28, 8691-8698

29. Galloway W A, Murphy G, Sandy J D, Gavrilovic J, Cawston T E, Reynolds J J. (1983) Biochem J 209, 741-752

30. Chin JR, Murphy G, Werb Z. (1985) J Biol Chem 260, 12367-12376

31. Okada Y, Nagase H, Harris E D Jr. (1986) J Biol Chem 261, 14245-14255

32. Formston C J, Byrd O J, Amrose H J, Riley J H, Hernandez D, McConville C M, Taylor A M. (1993) Genomics 16, 289-291

33. Rymaszewski Z, Cohen R M, Chomczynski P. (1991) Proc Natl Acad Sci USA 88, 617-621

34. Gould J, Aramburo C, Capdevielle M, Scanes C G. (1995) Life Sci 56, 587-594

35. Turner H E, Nagy Z, Gatter K C, Esiri M M, Harris A L, Wass J A. (2000) J Endocrinol 165, 475-481

36. Hellstrom A, Svensson E, Carlsson B, Niklasson A, Albertsson-Wikland K. (1999) 84, 795-798

37. Struman I, Bentzien F, Lee H, Mainfroid V, D'Angelo G, Goffin V, Weiner R I, Martial J A. (1999) Proc Natl Acad Sci USA 96, 1246-1251

38. Warner M D, Sinha Y N, Peabody C A. (1993) Horm Metab Res 25, 425-429

39. Young P K, Grinnell F. (1994) J Invest Dermatol 103, 660-664

40. Sacca L, Cittadini A, Fazio S. (1994) Endocr Rev 15, 555-573

41. Volterrani M, Giustina A, Manelli F, Cicoria M A, Lo russo R, Giordano A. (2000) Ital Heart J 1, 732-738

42. Friberg L, Werner S, Eggertsen G, Ahnve S. (2000) Eur Heart J 21, 1547-1554

43. Heymans S, Luttun A, Nuyens D, Theilmeier G, Creemers E, Moons L, Dyspersin G D, Cleutjens J P, Shipley M, Angellilo A, Levi M, Nube O, Baker A, Keshet E, Lupu F, Herbert J M, Smits J F, Shapiro S D, Baes M, Borgers M, Daemen M J, Carmeliet P. (1999) Nat Med 5, 1135-1142

44. Romanic A M, Burns-Kurtis C L, Berrebi-Bertrand I, Ohistein E H. (2001) Life Sci 68, 799-814

45. Kennedy T G, Doktorcik P E. (1988) Biol Reprod 39, 318-328

46. Hull K L, Harvey S. (2001) 168,1-23

47. Singtripop T, Mori T, Shiraishi K, Park M K, Kawashima S. (1993) In vivo 7, 147-150

48. Rodgers W H, Matrisian LM, Giudice L C, Dsupin B, Cannon P, Svitek C, Gorstein F, Osteen K G. (1994) J Clin Invest 94, 946-953

49. Norman J, Karasek M A. (1995) In Vitro Cell Dev Biol-Animal 31, 447-455

1. Method for cleaving the human growth hormone GH into two fragments of 16 kDa and 6 kDa, comprising using the human matrix metalloproteinase MMP-3, the 16 kDa fragment being stable and the 6 kDa fragment being less stable.

2. Method according to claim 1, wherein the recombinant catalytic domain of human MMP-3 effects the cleavage.

3. Method according to claim 1, wherein the full-length enzyme MMP-3 binds to human growth hormone GH and effects cleavage thereof.

4. Method according to claim 1, wherein the human growth hormone GH is cleaved by human MMP-3 in vitro.

5. A method for inhibiting tumor growth by lowering the level of human growth hormone GM in vivo comprising administering 3-(3-mercaptopropyl)-1-methyl-(1H,3H)quinazoline-2,4-dione and/or 3-(2′-mercaptoethylamino)-2H-1,2,4-benzothiadiazine-1,1-dioxide to a patient in need thereof.

6. A method for treating proliferative diabetic retinopathy comprising administering 3-(3-mercaptopropyl)-1-methyl-(1H,3H)quinazoline-2,4-dione and/or 3-(2′-mercaptoethylamino)-2H-1,2,4-benzothiadiazine-1,1-dioxide for the production of a pharmaceutical preparation to a patient in need thereof.

7. Preparation according to claim 5 wherein the pharmaceutical preparation contains conventional adjuvants and carriers and is administered by injection, applied as drops or given orally by means of tablets or capsules.

8. A method for the production of a pharmaceutical preparation for preventing the cleavage of human growth hormone GM by MMP-3, promoting angiogenesis, for the treatment of cardiac infarction, for the treatment of menstrual cycle disturbances and retarded wound healing comprising administering (R,S)-8-chloro-2-mercaptomethyl-2,3-dihydro-thiazolo[2,3b]quinazoline-5-one, (R,S)-1-(2′,3′-dimercapto-prop-1′-yl)quinazoline-2,4(1H,3H)-dione, 3-(2-mercaptopropyl)quinazoline-2,4 (1H,3H)-dione, (R,S)-2-mercaptomethyl-2-methyl-2,3-dihydro-thiazolo[2,3b]quinazoline-5-one and/or (R,S)-2-mercaptomethyl-2,3-dihydro-thiazolo[2,3b]quinazoline-5-one to a patient in need thereof.

9. The method according to claim 6, wherein it is administered as a pharmaceutical preparation in combination with conventional adjuvants and carriers by injection, applied as drops or given orally by means of tablets or capsules.

10. A method for preventing the cleavage of human growth hormone GH by MMP-3 thus for promoting angiogenesis, and for treating cardiac infarction, for treating disturbances of the menstrual cycle and retarded wound healing, comprising using compounds of the mercaptoalkylquinazoline class.

11. The method according to claim 10, comprising using (R,S)-8-chloro-2-mercaptomethyl-2,3-dihydro-thiazolo[2,3-b]quinazoline-5-one, (R,S)-1-(2′,3′-dimercapto-prop-1′-yl)quinazoline-2,4(1H,3H)-dione, 3-(2-mercaptopropyl)quinazoline-2,4 (1H,3H)-dione, (R,S)-2-mercaptomethyl-2-methyl-2,3-dihydro-thiazolo[2,3-b]quinazoline-5-one (R,S)-2-mercaptomethyl-2,3-dihydro-thiazolo[2,3-b]quinazoline-5-one.

12. The method according to claim 8, wherein the pharmaceutical preparation contains the compounds separately or in any admixture with one another by adding conventional adjuvants and carriers and is administered by injection, applied as drops or given orally by means of tablets or capsules.
